(19) United States
(12) Patent Application Publication
(10) Pub. No.: US 2015/0209595 A1
(43) Pub. Date: Jul. 30, 2015

(54) METHOD AND APPARATUS FOR INTRACELLULAR AND INTERCELLULAR DELIVERY OF MOLECULES, DRUGS, VACCINES AND THE LIKE

(71) Applicant: EP Technologies LLC, Akron, OH (US)

(72) Inventors: Sameer Kalghatgi, Copley, OH (US); Tsung-Chan Tsai, Cuyahoga Falls, OH (US); Daphne Pappas Antonakas, Hudson, OH (US); Robert L. Gray, Hudson, OH (US)

(21) Appl. No.: 14/610,467

(22) Filed: Jan. 30, 2015

Related U.S. Application Data
(60) Provisional application No. 61/933,384, filed on Jan. 30, 2014.

(51) Int. Cl. A61N 1/44; A61M 35/00

(52) U.S. Cl. CPC A61N 1/44; A61M 35/00

(57) ABSTRACT

An exemplary method of delivering drugs or vaccines includes applying a series of first electrical signals to an electrode to generate plasma. The first electrical pulses having a first duration, first voltage amplitude, and first rise time. Applying molecules, drugs or vaccines to an area of skin contacted by the plasma; and applying a series of second electrical signals to the electrode to generate plasma to contact the area of the skin. The second electrical pulses have a second duration, second voltage amplitude, and second rise time. The duration for the first electrical pulses is shorter than the duration for the second electrical pulses. The voltage amplitude of the second electrical pulses is larger than the first electrical pulses. The rise time of the second electrical pulses is shorter than the first electrical pulses.

### RELATED APPLICATIONS

This application claims priority to and the benefits of U.S. Provisional Patent Application Ser. No. 61/933,384 filed on Jan. 30, 2014 and entitled “METHOD AND APPARATUS FOR INTRACELLULAR AND INTERCELLULAR DELIVERY OF MOLECULES, DRUGS, VACCINES AND THE LIKE,” which is incorporated herein by reference in its entirety.

### BACKGROUND

Vaccines are one of the most important discoveries of modern medicine and the most beneficial treatment a physician can provide to a patient. Yet a number of vaccine preventable diseases await the technology to elicit the appropriate protective or therapeutic immune response. Most vaccines elicit antibody responses; however, cell mediated immune responses, including CD8 T cells are needed to prevent, control or treat intracellular bacterial, fungal and viral diseases as well as chronic diseases, including cancer.

DNA vaccines can obtain both cell mediated immune response and antibody responses. Accordingly, DNA vaccines represent an attractive alternative to other modes of vaccination. DNA vaccines consist of a plasmid (circle of DNA) containing the gene for the immunogenic protein necessary to elicit protection, proteins to enhance the immune response, and DNA sequences necessary for its transcription into RNA translation into protein in mammalian cells, and amplification in bacterial but not mammalian cells. The immune response to DNA vaccines resembles the response to a viral infection but is safer since DNA does not spread nor cause disease. DNA is also relatively easy to manufacture and stable to the environment. DNA vaccines may be used to generate the immune responses necessary to prevent or treat diseases, such as HSV, AIDS, hepatitis C, cancer and the like, that have eluded vaccine development by more conventional means.

Promoting efficient delivery and cellular uptake has been challenging and is the main reason that DNA vaccines have not been widely accepted yet. Several delivery methods for delivery and uptake of DNA vaccines including lipid mediated delivery, jet injections, gene guns and sonoporation, have been tested without much success.

Recent developments, in the field of DNA vaccine genetics and the use of electroporation for in vivo delivery of DNA vaccines, have increased efficiency of expression to levels that are practical in a real life setting. Electroporation uses pulsed electric currents to open pores and drive intradermally injected DNA into skin cells. Electroporation requires DNA injection in to the skin, direct electrode contact with skin and electric current application to promote cellular uptake of DNA. Electroporation as a drug delivery method has several drawbacks including pain, muscle contractions upon application and can cause current induced tissue damage. These drawbacks have limited its widespread adoption.

One study showed that the non-thermal plasma can also deliver pulsed electric fields to the skin and demonstrated that this method can safely promote cellular uptake of intradermally injected DNA vaccines. However, this method requires DNA to be injected into the skin with needles, which have negatives, such as, for example, they are painful and result in hazardous waste that must be disposed of. Further, an injection delivers a large quantity of the drug in a very localized area thereby limiting the interaction of the drug to a small number of cells and reducing the efficacy of treatment. Additionally, the study used a plasma jet which needs special equipment and expensive Helium gas. A further drawback of jets is the small surface area over which they can treat the skin.

Similarly, it may be desirable to promote cellular uptake of drugs, such as, for example, chemotherapeutic drugs, growth factors, immunomodulating drugs and the like without use of needles, which as noted above have a number of drawbacks.

### SUMMARY

An exemplary method of delivering drugs or vaccines includes applying a first electrical signal or a series of first electrical signals to an electrode to generate plasma over an area of skin, topically applying molecules, drugs or vaccines to an area of skin treated by the plasma; and applying a second electrical signal or a series of second electrical signals to the electrode to generate plasma over the same area of the skin. The duration for the first electrical pulse(s) is longer than the duration for the second electrical pulse(s).

Another exemplary method of delivering molecules, drugs or vaccines into cells includes applying a first electrical signal or a series of first electrical signals to an electrode to generate plasma over an area of skin tissue, topically applying molecules, drugs or vaccines to an area of skin treated by the plasma; and applying a second electrical signal or a series of second electrical signals to the electrode to generate plasma over the same area of the tissue. The first electrical signal(s) allows the drugs or vaccines to move intercellularly (around the cells) and the second electrical signal(s) causes the drugs or vaccines to move intracellularly (in to the cells).

An exemplary apparatus for delivering molecules, drugs or vaccines intercellularly and intracellularly includes a plasma generating device and a power supply for powering the plasma generating device. Circuitry for providing a first electrical pulse or a series of first electrical pulses to the plasma generating device and circuitry for providing a second electrical pulse or a series of second electrical pulses to the plasma generating device are also included. In addition, a reservoir containing one or more molecules, drugs or vaccines are provided. The first electrical pulse(s) causes one or more molecules to pass through layers of skin or tissue and the second electrical pulse(s) causes the one or more molecules to pass into one or more cells in the skin or tissue.

Another exemplary apparatus for delivering molecules drugs or vaccines intercellularly and intracellularly includes a plasma generating device, a power supply for powering the plasma generating device. In addition, the apparatus includes intercellular poration circuitry for causing at least one of molecules, drugs or vaccines through pores in skin or tissue that are between cells. Intracellular poration circuitry for causing the at least one of molecules, drugs or vaccines into cells is also included. The apparatus may include a reservoir containing one or more molecules, drugs or vaccines to be driven intercellularly and then intracellularly.

### BRIEF DESCRIPTION OF THE DRAWINGS

These and other features and advantages of the present invention will become better understood with regard to the following description and accompanying drawings in which:

FIG. 1 is a schematic view of an exemplary embodiment of an apparatus for intercellular and intracellular poration shown in an intercellular poration configuration;

FIG. 2 is an cross-section showing layers of the skin and exemplary intercellular paths for molecules, drugs, vaccines and the like;

FIG. 3 is a schematic view of an exemplary embodiment of an apparatus for intercellular and intracellular poration in a intracellular poration configuration;

FIG. 4 is an cross-section showing layers of the skin and exemplary intracellular paths for molecules, drugs, vaccines and the like;

FIG. 5 is a schematic view of another exemplary embodiment of an apparatus for intracellular and intracellular poration; and

FIG. 6 is a block diagram of an exemplary methodology for intercellular and intracellular poration.

### DETAILED DESCRIPTION

Applicants have developed techniques for moving molecules, drugs, DNA and the like across layers of the skin, both intercellularly (through the skin) and intracellularly (in to the cells) using plasma. Applicants filed U.S. Provisional Application Ser. No. 61/883,701 filed on Sep. 27, 2013 and U.S. Non-Provisional application Ser. No. 14/500,144, filed on Sep. 29, 2014, both of which are entitled Method and Apparatus for Delivery of Molecules Across Layers of the Skin, and both are incorporated herein by reference in their entirety. Applicants' exemplary methods utilize plasma for providing a safe, contact-less delivery and cellular uptake of DNA vaccines, which may be referred to herein as plasmaporation. Applicants also filed U.S. Provisional Application Ser. No. 61/911,536 filed on Dec. 4, 2013 and U.S. Non-Provisional application Ser. No. 14/560,343 filed on Dec. 4, 2014, both of which are entitled Transdermal Delivery of DNA Vaccines Using Non-Thermal Plasma, and are both incorporated herein by reference in their entirety.

Plasmaporation uses non-thermal plasma, the fourth state of matter, for transdermal delivery of molecules, drugs, vaccines and the like through tissue and into cells. Non-thermal plasma is a partially ionized gas generated at atmospheric pressure using electricity. It is generated by the breakdown of air or other gases present between two electrodes under the application of sufficiently high voltage. The pulsed electric field used to generate the plasma opens up temporary pores in the skin and within cells to promote transdermal delivery and cellular uptake of molecules (including macromolecules), drugs, vaccines and the like. In some embodiments, the temporary pores remain open for about 1 to about 5 minutes.

The electrodes are not in contact with the skin, no needles are required and generation of non-thermal plasma directly on skin is rapid and painless. In exemplary embodiments with configurations where the electrodes are insulated, non-thermal plasma is formed by dielectric barrier discharge (DBD), which is safe and painless when applied to skin. The plasmaporation technique described herein is a more efficient and rapid means of delivery in a painless manner without the need for injection. Accordingly, the plasmaporation technique described herein can promote efficient intercellular delivery and intracellular uptake of molecules, drugs, vaccines, and the like.

In some exemplary embodiments, plasmaporation involves the use of a planar DBD or a DBD jet plasma generator for needle-free transdermal delivery of macromolecules. Depending on the plasma dose, the depth of penetration of the macromolecules can be regulated to ensure delivery to the target layer (stratum corneum, epidermis and dermis).

Applicants have demonstrated that plasmaporation can enhance transdermal delivery of topically applied dextran molecules with molecular weights up to 70 kDa across ex vivo porcine skin within 15 minutes and without creating skin damage, as described in the patent applications entitled Method and Apparatus for Delivery of Molecules Across Layers of the Skin on Sep. 27, 2013 and Sep. 29, 2013 incorporated herein.

In the plasma phase, neutral gas atoms (or molecules), electrons, positive/negative ions, and radicals are generated. Their generation and concentration depend, in part, on the physical and chemical properties of the gas being used to generate the plasma as well as the electrical parameters used to generate the plasma. The strength of the electric field generated by non-thermal plasma on skin can be tuned by varying the time of plasma treatment; gap between the electrode and the skin; applied voltage; pulse duration; frequency and duty cycle to localize delivery. These parameters allow control of the depth and delivery amount of macromolecules, drugs, vaccines and the like across the skin allowing treatment of the targeted skin layer with an optimal dose.

The exemplary embodiments of apparatuses and method disclosed herein use non-thermal plasmas to enable transdermal delivery of macromolecules, drugs, vaccines and the like, through the surface and in to ex vivo porcine skin without harming the skin. Non-thermal plasma enabled skin poration provides a non-invasive, safe means for transdermal delivery and cellular uptake of molecules, drugs and vaccines at room temperature and atmospheric pressure without the possible pain and other side effects associated with electroporation. As the application of the method does not require disposable electrodes or needles, the need for disposal of biohazardous waste and illicit use of biohazardous consumables is eliminated. An additional benefit of using non-thermal plasma is that the generated reactive species sterilizes the skin during plasmaporation.

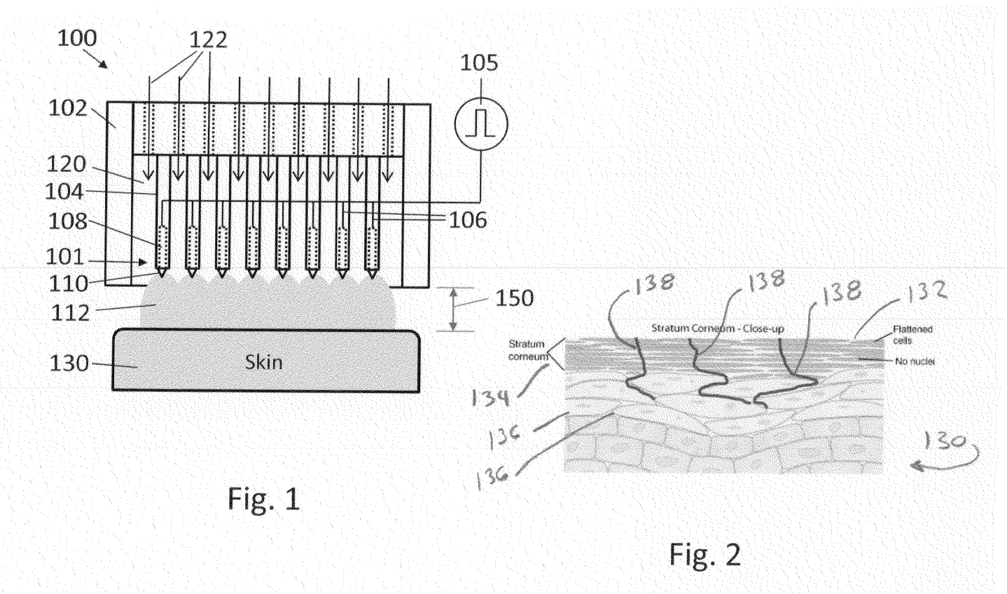

FIG. 1 is a schematic view of an exemplary embodiment of an apparatus 100 for intercellular and intracellular poration set up in an intercellular poration configuration. The apparatus 100 include a housing 102. A plurality of plasma generators 101 is located within the housing. In some embodiments, plasma generators 101 are arraigned in a one-dimensional array. In some embodiments, plasma generators 101 are arraigned in a two-dimensional array. In some embodiments, plasma generators 101 are arranged in a three-dimensional array. Each plasma generator 101 includes an insulator 104, such as for example, fused quartz glass, magnesium fluoride, aluminum nitrate, aluminum nitrite, TEFLON® (polytetrafluoroethylene), aluminum oxide, alumina, silicate, or the like. Located within the insulator 104 is a plurality of electrodes 108. In some embodiments, the electrodes 108 have exposed tips 110 for plasma 112 generation. In some embodiments, the electrodes 108 are copper. Optionally, electrodes 108 may be, for example, titanium, silver, aluminum, gold, metal alloys, carbon nanofibers, carbon nanowires or other conductive materials. A plurality of electrical conductors 106 connects the electrodes 108 to a high voltage power source 105. In some embodiments, the high voltage power source 105 is a power supply, which can produce high-voltage pulses with pulse duration ranging from one or more nanoseconds to one or more microseconds. In some embodiments, the power supply operates at frequencies ranging from single pulse to about 5 kHz. In some embodiments, the voltage amplitude ranges from between about 100 V to about 30 kV.

During intercellular poration, control circuitry (not shown) causes the high voltage power source 105 to apply one or more long voltage pulses at moderate amplitudes with moderate rise times. In some embodiments, the long pulses are between about 100 nanoseconds and 100 microseconds. In some embodiments the moderate amplitude is between about 3 kilovolts to about 30 kilovolts, and in some embodiments between about 3 to about 10 kilovolts. In some embodiments the moderate rise time is between about 5 V/ns to about 100 V/ns.

In some embodiments, plasma is applied in a dynamic mode. In some embodiments the plasma is provided in a static mode, and in some embodiments, plasma is applied in both a dynamic mode and a static mode. The dynamic mode is when the plasma will be applied in a predetermined pattern or motion over area to be treated. One predetermined pattern or motion may be, for example, a sweeping motion. The sweeping motion may be accomplished by moving the electrodes 108 along the surface to be treated. In some embodiments an array of electrodes are used and the sweeping motion is accomplished by sending signals to selected electrodes in a sweeping pattern. A static mode is when the electrodes are kept in a fixed position with respect to the surface being treated and energized at the same time. In some embodiments, the dynamic mode is used for driving the molecules, particles, vaccines and the like intercellularly and the static mode is used for driving the molecules, particles, vaccines and the like intracellularly. In some embodiments, the static mode is used for driving the molecules, particles, vaccines and the like intercellularly and the dynamic mode is used for driving the molecules, particles, vaccines and the like intracellularly. In some embodiments the static mode is used for driving the molecules, particles, vaccines and the like intercellularly and intracellularly. In some embodiments the dynamic mode is used for driving the molecules, particles, vaccines and the like intercellularly and intracellularly.

Housing 102 includes a plurality of passages 120. Passages 120 allow a gas 122 to flow through the housing 102 to an area below electrodes 108. The gas 122 may be used to alter the property of the plasma 112 being generated by electrodes 108 when a high voltage is applied to the electrodes 108. Electrodes 108 may take various shapes. In some embodiments electrodes 108 may be sharp tipped conductive wires and in some embodiments electrodes 108 may be wires having a diameter of about 0.05 mm to about 3 mm. In some embodiments, the gas 122 is helium. In some embodiments, the gas 122 is an inert gas. In some embodiments, the gas 122 is a noble gas. In some embodiments the gas 122 is He, Ne, Ar, Xe, or the like. In some embodiments, the gas 122 is a mixture of gases that may include one or more inert gases or noble gases. In some embodiments, the gas 122 is a gas, which can sustain plasma 112 for about 100 nanoseconds to about 100 microseconds. In some embodiments, the plasma 112 is corona discharge. In some embodiments, additives, such as, for example, ethanol, water vapor, etc. may be added to the gas 122. In some embodiments, the electrodes 108 are covered by a plurality of insulators 104 with exposed tips 110. Housing 102 is spaced above skin 130 by a distance 150. In some embodiments, distance 150 is between about 1 mm and about 10 mm.

In some embodiments, molecules, drugs, vaccines, or the like may be combined with gas 122 to be applied to a treatment area. In some embodiments, gas 122 is used in the generation of plasma, the plasma generators 101 are turned off, and molecules, drugs, vaccines, or the like are applied to the surface of the skin through passages 120. In some embodiments, apparatus 100 is removed after treating the surface of the skin 130 with plasma and the molecules, drugs, vaccines, or the like are applied to the skin 130. In some embodiments, after the molecules, drugs, vaccines, or the like are applied to the surface of the skin 130, apparatus 100 is again operated with the intercellular setting identified above to help drive the molecules, drugs, vaccines, or the like through the stratum corneum 134 (FIG. 2) which includes a layer of flattened cells with no nuclei and between cells 136 that contain nuclei. The long duration pulses and moderate amplitudes drive the molecules, drugs, vaccines, or the like intercellularly through the exemplary intercellular paths 138.

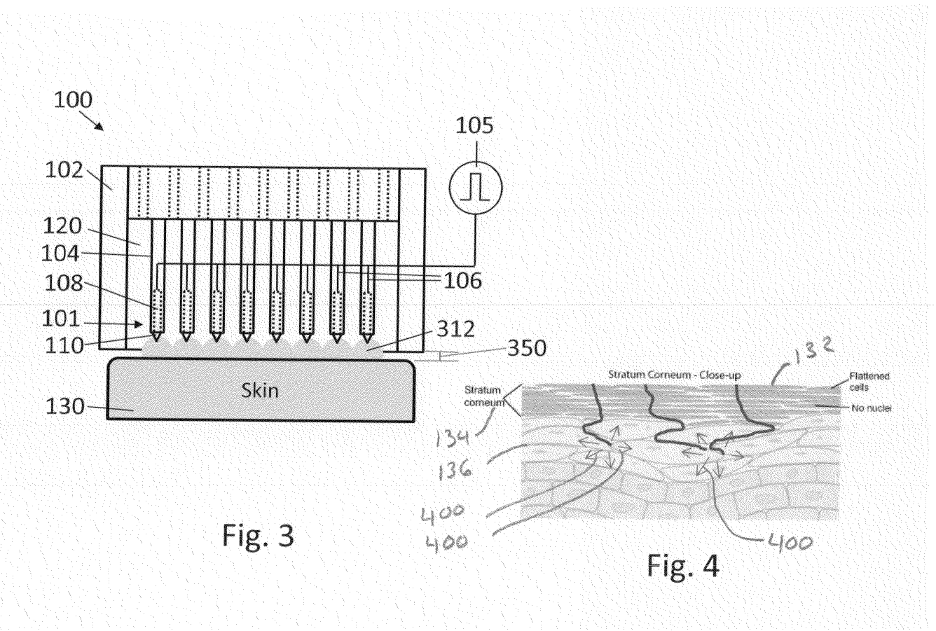

FIG. 3 is a schematic view of the exemplary embodiment of apparatus 100 in an intracellular poration configuration. Housing 102 is located a distance 350 from skin 130. In some embodiments, distance 350 is between about 1 mm and 5 mm. In one exemplary embodiment, plasma 312 is created in atmospheric air. The atmospheric air may be ambient air, dry or humid, located below housing 102, or optionally be air passed through passages 120. In some embodiments, the gas is a nitrogen gas. In some embodiments, the gas is a gas, which can only sustain plasma 312 for between about 1 nanosecond to about 100 nanoseconds. In some embodiments, the plasma 312 is corona discharge. During intracellular poration, the power supply provides short duration pulses with high amplitudes with fast rise times. In some embodiments, the short duration pulses are between about 1 nanosecond and 100 nanoseconds. In some embodiments, the high amplitude is between about 10 kilovolts and about 30 kilovolts. In some embodiments the fast rise time is between about 0.5 kV/ns to about 5 kV/ns. The short duration pulses with high amplitudes and fast rise times cause the molecules, drugs or vaccines to be driven into the cells due to the creation of temporary pores in the cell membranes.

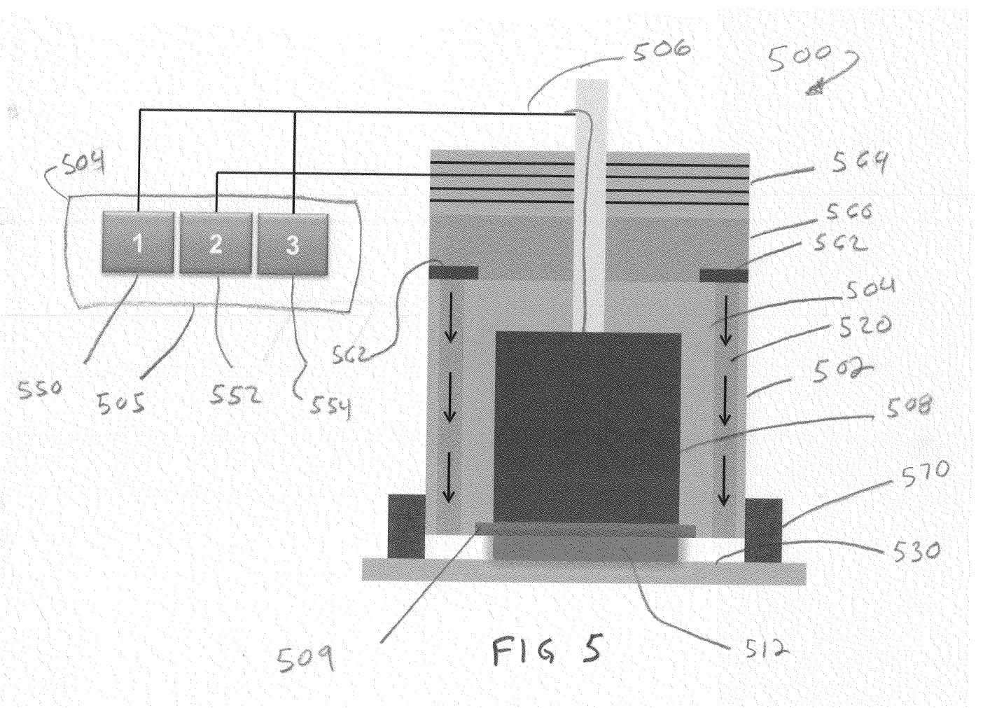

FIG. 4 is an cross-section showing layers of the skin 130 and exemplary intercellular paths 138 for molecules, drugs, vaccines, or the like and the intracellular paths 400 for the molecules, drugs, vaccines or the like into cells 136.

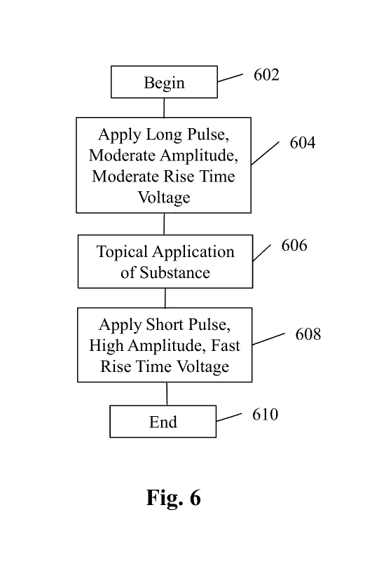

FIG. 5 is a schematic view of another exemplary embodiment of an apparatus 500 for intercellular and intracellular poration. Apparatus 500 includes a housing 502. An electrode 508 is located within an insulator 504. Electrode 508 and insulator 504 may be made of the similar materials to those identified above. A dielectric barrier 509 is below electrode 508. Attached to housing 502 are one or more spacers 570. Spacers 570 create a gap between dielectric barrier 509 and the surface of the skin 530. In some embodiments, spacers 570 are adjustable and may be adjusted to a first range of heights for intercellular poration and a second range for intracellular poration. In some embodiments, the spacer includes a grounding conductor (not shown) to provide a ground path back to apparatus 500.

Apparatus 500 includes control circuitry 504. Control circuitry 504 includes intercellular poration circuitry 550 and intracellular poration circuitry 554. Electrode 508 is in circuit communication with intercellular poration circuitry 550 and intracellular poration circuitry 554.

Although the electrical components are described as being in certain locations, or as being part of an “electronics package,” the components may be located in any suitable location and more or less components may be included. The term electronics package is merely used for convenience and is not meant to limit the number of components or their location.

“Circuit communication” as used herein indicates a communicative relationship between devices. Direct electrical, electromagnetic and optical connections and indirect electrical, electromagnetic and optical connections are examples of circuit communication. Two devices are in circuit communication if a signal from one is received by the other, regardless of whether the signal is modified by some other device. For example, two devices separated by one or more of the following—amplifiers, filters, transformers, optoisolators, digital or analog buffers, analog integrators, other electronic circuitry, fiber optic transceivers or satellites—are in circuit communication if a signal from one is communicated to the other, even though the signal is modified by the intermediate device(s). As another example, an electromagnetic sensor is in circuit communication with a signal if it receives electromagnetic radiation from the signal. As a final example, two devices are not directly connected to each other, but both capable of interfacing with a third device, such as, for example, a CPU, are in circuit communication.

Also, as used herein, voltages and values representing digitized voltages are considered to be equivalent for the purposes of this application, and thus the term “voltage” as used herein refers to either a signal, or a value in a processor representing a signal, or a value in a processor determined from a value representing a signal.

“Signal”, as used herein includes, but is not limited to one or more electrical signals, analog or digital signals, one or more computer instructions, a bit or bit stream, or the like.

“Logic,” synonymous with “circuit” as used herein includes, but is not limited to hardware, firmware, software and/or combinations of each to perform a function(s) or an action(s). For example, based on a desired application or needs, logic may include a software controlled microprocessor or microcontroller, discrete logic, such as an application specific integrated circuit (ASIC) or other programmed logic device. Logic may also be fully embodied as software. The circuits identified and described herein may have many different configurations to perform the desired functions.

The values identified in the detailed description are exemplary and they are determined as needed for a particular design. Accordingly, the inventive concepts disclosed and claimed herein are not limited to the particular values or ranges of values used to describe the embodiments disclosed herein.

Intercellular poration circuitry 550 includes circuitry for providing long pulses having moderate amplitudes with moderate rise times. In some embodiments, the long pulses are between about 100 nanoseconds and about 100 microseconds. In some embodiments the moderate amplitude is between about 3 kilovolts to about 30 kilovolts and in some embodiments is between about 3 kilovolts to about 10 kilovolts. In some embodiments the moderate rise time is between about 5 V/ns to about 100 V/ns. The long duration pulses with moderate amplitudes and moderate rise times cause the molecules, drugs or vaccines to be driven through the tissue between cells via intercellular poration.

Intracellular poration circuitry 554 includes circuitry for providing short pulses at high amplitudes with fast rise times. In some embodiments, the short duration pulses are between about 1 nanosecond and about 100 nanoseconds. In some embodiments, the high amplitude is between about 10 kilovolts and about 30 kilovolts. In some embodiments the fast rise time is between about 0.5 kV/ns to about 10 kV/ns and in some embodiments is between about 0.5 kV/ns to about 5 kV/ns. The short duration pulses with high amplitudes and fast rise times cause the molecules, drugs or vaccines to be driven into the cells because of intracellular poration.

Control circuitry 504 also includes delivery circuitry 552 for delivering molecules, drugs, vaccines, nanoparticles, encapsulated molecules, and the like to the surface of the skin. Housing 502 includes a reservoir 560 for holding molecules, drugs, vaccines, nanoparticles, encapsulated molecules and the like. In addition, housing 502 includes passages 520 between reservoir 560 and the surface of the skin 530. One or more valves 562 are located upstream of passage 520. In addition, an actuator 564 is located proximate to reservoir 560 to push the molecules, drugs or vaccines out of the reservoir 560. During operation, when it is time to deliver the molecules, drugs, vaccines, nanoparticles, delivery vehicles, encapsulated molecules, or the like to the surface of the skin, delivery circuitry opens the one or more valves 562 and reduces the volume of reservoir 560 to cause the molecules, drugs or vaccines to reach the surface of the skin.

During operation, in some embodiments, such as, for example, when used for DNA vaccines, intercellular poration circuitry 550 is activated to induce formation of temporary pores (poration) between the flat cells of the stratum corneum and between cells having a nucleus. Delivery circuitry 552 is activated to deliver the vaccine to the surface of the skin 530. Once the vaccine is applied to the surface of the skin 530, intracellular poration circuitry 554 is activated to cause the vaccine to be driven into the cells. In some embodiments, the vaccine is applied to the surface of the skin 530 before the intercellular poration circuitry is activated. In some embodiments, the intercellular poration circuitry 550 is activate before and after the delivery circuitry 552 is activated.

In some embodiments, such as, for example, drug delivery, intercellular circuitry 550 may be activated to open pores in the skin and delivery circuitry 552 may be activated to apply drugs to the surface of the skin 530. In some embodiments, the above steps may be followed by a second activation of intercellular circuitry 550. In some embodiments, delivery circuitry 552 may be activated to apply drugs to the surface of the skin 530 and then intercellular circuitry 550 may be activated to drive the drug through pores between the cells.

In some embodiments, housing 502 may include a second passageway (not shown) for applying a gas, such as, for example, helium, to the area between the skin 530 and electrode 508 for altering the properties of the plasma generated by the high voltage pulses.

Although the embodiments described herein are described with respect to skin, the inventive concepts described herein are applicable to other tissue or organs. In addition, while molecules, drugs and vaccines have been particularly called out, particles, the exemplary applications described herein are applicable to DNA vaccines, to application of growth factors, antitumor drugs, chemotherapeutic drugs, immunomodulating drugs, particles and the like where it may be desirable to move the item between cells, such as those in the stratum corneum and then into cells, such as those in the epidermis or dermis.

FIG. 6 is a block diagram of an exemplary methodology for intercellular and intracellular poration. The exemplary methodology may be carried out in logic, software, hardware, or combinations thereof. In addition, although the methodology is presented in an order, the blocks may be performed in different orders. Further, additional steps or fewer steps may be used.

The exemplary methodology 600 begins at block 602. At block 604, a long voltage pulse having a moderate amplitude and moderate a rise time is applied to generate plasma for creating temporary intercellular pores. At block 606, molecules, drugs or vaccines are applied to the tissue. The molecules, drugs or vaccines travel through the pores between the cells. In some embodiments, the long voltage pulse is reapplied to drive the molecules, drugs or vaccines through the pores. At block 608, a short pulse voltage having a high amplitude and a fast rise time is applied to the electrode to create plasma that drives the molecules, drugs or vaccines into the cells via formation of temporary pores in cell membranes. The methodology ends at block 610.

Another benefit of the exemplary embodiments disclosed herein is plasmaporation of the stratum corneum for intercellular poration may create or open a large number of pores, indeed depending on the design of the electrodes, millions and millions of pores may be created. Injected vaccines or molecules are concentrated at one or more needle injection sites, whereas the topical applications of vaccines or molecules as disclosed herein may be located at each created or opened pore. According, rather than having the dose of vaccine or molecules concentrated at injection locations, the number of discrete cites that the same volume of vaccine or molecules may be increased exponentially. Although this paragraph discusses vaccines and molecules, the exemplary methodologies work for other chemicals, molecules, nonparties, encapsulated molecules, and the like. The only limitation is the substance needs to fit through the created or opened pores.

A number of experiments were conducted on live animals. Five to seven month old Yucatan minipigs were utilized in live animal experiments. Experimental controls included: plasmid DNA injected intradermally with no following treatment; and plasmid DNA injected intradermally followed by electroporation (current state of the art). Experimental samples included: intradermal injection of plasmid DNA followed by microsecond plasma after; intradermal injection of plasmid DNA followed by nanosecond plasma; intradermal injection of plasmid DNA followed by corona array plasma; microsecond plasma followed by topical plasmid DNA application followed by microsecond plasma; microsecond plasma followed by topical plasmid DNA application followed by nanosecond plasma; nanosecond plasma followed by topical plasmid DNA application followed by nanosecond plasma; corona array plasma followed by topical plasmid DNA application followed by corona array plasma; and nanosecond plasma followed by topical plasmid DNA application followed by nanosecond plasma.

The Chart below provides the experimental results. The first column is titled Sample, and identifies whether the experiment was a straight control or an electroporation control experiment or a plasma treatment experiment. “Treatment” indicates plasma treatment data. “Control” indicates that the data is control data, and “EP” indicates electroporation control data. Column 2 titled “Delivery” indicates whether the molecules were injected into the skin or whether they were topically applied. Column 3 identifies the power supply used. Columns 4-11 identify the settings used. Column 11 indicates the raw expression data. Column 12 indicates normalized expression data, which was determined by subtracting the intensity of fluorescent signal from skin that did not receive any DNA and was not plasma treated. The last column, column 13 identifies the percentage increase in expression in the plasma treated or electroporated samples over the injected control with no follow up treatment.

| | | | | | | Pulse | | | | | | | | % |
|---|---|---|---|---|---|---|---|---|---|---|---|---|---|---|
| | | | | | fre- | dura- | Duty | Volt- | | | Hold | | | Increase |
| | | | Power | | quency | tion | Cycle | age | # | Time | Time | Raw | Normalized | over |
| | Sample | Delivery | Supply | Mode | (Hz) | (μs) | (%) | (kV) | Pulses | (s) | (s) | expression | Expression | injected |
| 1 | Treatment | Injected | microsecond | continuous | 3500 | 5 | 100 | 20 | — | 30 | — | 1.87E+07 | 5.01E+06 | 156% |
| 2 | Treatment | injected | nanosecond | pulsed | — | 0.5 | — | 20 | 25 | — | — | 1.90E+07 | 5.29E+06 | 170% |
| 3 | Treatment | injected | nanosecond | continuous | 200 | 0.2 | — | 20 | — | 120 | — | 1.86E+07 | 4.89E+06 | 150% |
| 4 | control | Injected | | | | | | | | | | 1.566E+07 | 1.956E+06 | |
| 5 | EP | injected | | | | | | | | | | 1.784E+07 | 4.134E+06 | 111% |
| 1 | Treatment | Injected | ns corona | pulsed | — | 0.1 | — | 20 | 25 | — | — | 3.13E+07 | 8.61E+06 | 117% |
| | | | array | | | | | | | | | | | |
| 2 | Treatment | Injected | ns corona | continuous | 100 | 0.08 | — | 20 | | 30 | — | 3.05E+07 | 7.82E+06 | 97% |
| | | | array | | | | | | | | | | | |
| 3 | control | Injected | | | | | | | | | | 2.668.E+07 | 3.960E+06 | |
| 4 | EP | injected | | | | | | | | | | 2.802.E+07 | 4.295E+06 | 33% |
| 1 | Treatment | topical | microsecond | continuous | 3500 | 5 | 100 | 15 | — | 90 | 60 | 8.86E+07 | 1.46E+07 | 33% |
| | | | microsecond | continuous | 3500 | 10 | 100 | 20 | — | 60 | — | | | |
| 2 | Treatment | topical | microsecond | continuous | 3500 | 5 | 100 | 15 | — | 90 | 60 | 9.00E+07 | 1.59E+07 | 46% |
| | | | microsecond | continuous | 3500 | 10 | 100 | 20 | — | 60 | — | | | |
| 3 | Treatment | topical | microsecond | continuous | 3500 | 10 | 100 | 20 | — | 60 | 60 | 9.88E+07 | 2.47E+07 | 126% |
| | | | nanosecond | continuous | 500 | 0.5 | — | 20 | — | 30 | — | | | |
| 4 | Treatment | topical | microsecond | continuous | 3500 | 10 | 100 | 20 | — | 60 | 60 | 1.00E+08 | 2.63E+07 | 140% |
| | | | nanosecond | pulsed | — | 0.5 | — | 20 | 25 | — | — | | | |
| 5 | control | injected | | | | | | | | | | 8.60E+07 | 1.19E+07 | |
| | | | | | | | | | | | | 8.40E+07 | 9.96E+06 | |
| 6 | EP | injected | | | | | | | | | | 9.44E+07 | 2.04E+07 | 86% |
| | | | | | | | | | | | | 9.30E+07 | 1.90E+07 | 73% |
| 1 | Treatment | Topical | corona array | pulsed | — | 0.04 | — | 20 | 25 | — | 60 | 1.02E+08 | 1.89E+07 | 35% |
| | | | corona array | pulsed | — | 0.08 | — | 15 | 25 | — | — | | | |
| 2 | Treatment | Topical | corona array | pulsed | — | 0.04 | — | 20 | 25 | — | 60 | 9.89E+07 | 1.54E+07 | 10% |
| | | | corona array | pulsed | — | 0.08 | — | 15 | 25 | — | — | | | |
| 3 | Treatment | Topical | nanosecond | continuous | 1000 | 0.5 | — | 15 | — | 60 | 60 | 1.03E+08 | 1.95E+07 | 39% |
| | | | nanosecond | continuous | 500 | 0.5 | — | 20 | — | 50 | — | | | |
| 4 | Treatment | Topical | nanosecond | continuous | 1000 | 0.5 | — | 15 | — | 50 | 60 | 1.01E+08 | 1.75E+07 | 25% |
| | | | nanosecond | pulsed | — | 0.5 | — | 20 | 25 | — | — | | | |
| 5 | control | injected | | | | | | | | | | 1.00E+08 | 1.65E+07 | |
| | | | | | | | | | | | | 9.50E+07 | 1.15E+07 | |
| 6 | EP | injected | | | | | | | | | | 1.08E+08 | 2.43E+07 | 74% |
| | | | | | | | | | | | | 1.01E+08 | 1.80E+07 | 28% |

As can be seen from the chart, microsecond pulsed plasma followed by topical application followed by microsecond pulsed plasma had a greater efficacy than the injected control. It is believed that optimizing the settings of the power supply will increase the efficacy. Similarly, corona array pulsed plasma followed by topical treatment, followed by corona array plasma had a greater efficacy than the injected control. It is believed that optimizing the settings of the power supply will increase the efficacy. Similarly, nanosecond pulsed plasma followed by topical treatment followed by nanosecond pulsed plasma had a greater efficacy than the injected control. It is believed that optimizing the settings of the power supply will increase the efficacy.

The experimental results demonstrated that microsecond pulsed plasma followed by topical treatment followed by nanosecond pulsed plasma had very good efficacy. It is believed that optimizing the settings of the power supply will increase the efficacy with this methodology as well.

While the present invention has been illustrated by the description of embodiments thereof and while the embodiments have been described in considerable detail, it is not the intention of the applicant to restrict or in any way limit the scope of the appended claims to such detail. Additional advantages and modifications will readily appear to those skilled in the art. For example, Flexible and wearable electrodes may be developed and the generation of the non-thermal plasma can be optimized for transdermal delivery. The methods described herein may be used to cause cellular uptake of other macromolecules (e.g. antibodies, drugs) in addition to DNA vaccines. Therefore, the invention, in its broader aspects, is not limited to the specific details, the representative apparatus and illustrative examples shown and described. Accordingly, departures may be made from such details without departing from the spirit or scope of the applicant's general inventive concept.

We claim:

1. A method of delivering molecules, particles, drugs or vaccines comprising:
applying one or more first electrical pulses to an electrode to generate plasma;
the first electrical pulses having a first duration;
applying molecules, drugs or vaccines to an area of skin contacted by the plasma; and
applying one or more second electrical pulses to the electrode to generate plasma proximate the area of the skin;
the second electrical pulses having a second duration;
wherein the duration for the one or more first electrical pulses are longer than the duration for the one or more second electrical pulses.

2. The method of claim 1 wherein the duration of the one or more first electrical pulses is between about 500 nanoseconds and about 100 microseconds.

3. The method of claim 1 wherein the amplitude of the one or more first electrical pulses is between about 3 kilovolts and about 30 kilovolts.

4. The method of claim 1 wherein the rise time of the one or more first electrical pulses is between about 5 V/ns and about 100 V/ns.

5. The method of claim 1 wherein the duration of the one or more second electrical pulses is between about 1 nanosecond and about 500 nanoseconds.

6. The method of claim 1 wherein the duration of the one or more second electrical pulses is less than about 10 nanoseconds.

7. The method of claim 1 wherein the duration of the second electrical pulses is less than about 500 nanoseconds.

8. The method of claim 1 wherein the amplitude of the one or more second electrical pulses is between about 10 kilovolts and about 30 kilovolts.

9. The method of claim 1 wherein the rise time of the one or more second electrical pulses is between about 0.5 kV/ns and about 10 kV/ns.

10. The method of claim 1 wherein the drugs or vaccines are applied after one of the one or more first electrical pulses.

11. The method of claim 1 wherein the distance between the electrode and the skin during the one or more first electrical pulses is greater than the distance between the electrode and the skin during the one or more second electrical pulses.

12. A method of delivering drugs or vaccines into cells comprising:
applying one or more first electrical signals to an electrode to generate plasma on an area of skin tissue;
applying drugs or vaccines to an area of skin contacted by the plasma; and
applying one or more second electrical signals to the electrode to the area of the tissue;
wherein the one or more first electrical signals allows the drugs or vaccines to move intercellularly and
wherein the one or more second electrical signals causes the drugs or vaccines to move intracellularly.

13. The method of claim 12 wherein the drugs or vaccine are applied after one of the one or more first electrical signals.

14. The method of claim 12 wherein the distance between the electrode and the skin during the one or more first electrical signals is greater than the distance between the electrode and the skin during the one or more second electrical signals.

15. An apparatus for delivering molecules, particles, drugs or vaccines intercellularly and intracellularly comprising:
a plasma generating device;
a power supply for powering the plasma generating device;
circuitry for providing one or more first electrical pulses to the plasma generating device;
circuitry for providing one or more second electrical pulses to the plasma generating device;
a reservoir containing one or more molecules, particles, drugs or vaccines;
wherein the one or more first electrical pulses cause the one or more molecules, particles, drugs or vaccines to pass through layers of tissue; and
the one or more second electrical pulses cause the one or more molecules, particles, drugs or vaccines to pass into one or more cells in the tissue.

16. An apparatus for delivering molecules, particles, drugs or vaccines intercellularly and intracellularly comprising:
a plasma generating device;
a power supply for powering the plasma generating device;
intercellular poration circuitry for causing at least one of molecules, drugs or vaccines through pores in tissue that are between cells;
intracellular poration circuitry for causing the at least one of molecules, drugs or vaccines into cells; and
a reservoir containing one or more molecules, drugs, or vaccines.

17. The apparatus of claim 16 wherein the plasma generating device comprises a housing and the reservoir is located within the housing.

18. The apparatus of claim 16 further comprising one or more spacers for spacing the plasma generator away from a surface.

19. The apparatus of claim 18 wherein the one or more spacers are adjustable and may be set at a first height during use of the intercellular poration circuitry and a second height during use of the intracellular poration circuitry.

20. The apparatus of claim 16 further comprising a source of gas proximate to the plasma generating device.
